SEQUENCE LISTING (10 sequences)

SEQ ID NO: 1 - DNA, 21 bp, Homo sapiens
ccgaatattcttacaagctcc

SEQ ID NO: 2 - DNA, 20 bp, Homo sapiens
ggacctgtttttaggtactg

SEQ ID NO: 3 - DNA, 20 bp, Homo sapiens
agccacatcgctcagacacc

SEQ ID NO: 4 - DNA, 19 bp, Homo sapiens
gtactcagcgccagcatcg

SEQ ID NO: 5 - DNA, 20 bp, Mus musculus
cacaaaacatctccagtcac

SEQ ID NO: 6 - DNA, 21 bp, Mus musculus
tcttcgtcgttatagtgtctc

SEQ ID NO: 7 - DNA, 20 bp, Mus musculus
aaggtcatcccagagctgaa

SEQ ID NO: 8 - DNA, 20 bp, Mus musculus
ctgcttcaccaccttcttga

SEQ ID NO: 9 - DNA, 18 bp, Homo sapiens
tctcccaacccttgtacc

SEQ ID NO: 10 - DNA, 18 bp, Homo sapiens
acacgcaaattcgtgaag

(19) United States
(12) Patent Application Publication
(10) Pub. No.: US 2022/0202951 A1
(43) Pub. Date: Jun. 30, 2022

(54) LIGHT-TRIGGERABLE NANOPARTICLE LIBRARY OF FORMULATIONS FOR THE CONTROLLED RELEASE OF RNAS

(71) Applicants: UNIVERSIDADE DE COIMBRA, Coimbra (PT); CENTRO DE NEUROCIENCIAS E BIOLOGIA CELULAR, Coimbra (PT)

(72) Inventors: Lino DA SILVA FERREIRA, Coimbra (PT); Josephine BLERSCH, Coimbra (PT)

(21) Appl. No.: 17/611,278

(22) PCT Filed: May 14, 2020
(86) PCT No.: PCT/IB2020/054585

§ 371 Date: Nov. 15, 2021

(30) Foreign Application Priority Data
May 14, 2019 (PT) 115513

(51) Int. Cl. A61K 47/69; A61K 41/00; A61K 9/51

(52) U.S. Cl. CPC A61K 47/6901; A61K 41/0028; A61K 9/5146; A61K 47/6909; A61K 41/0042

(57) ABSTRACT

A triggerable polymeric nanoparticle (NP) library composed by several formulations, presenting physico-chemical diversity and differential responsiveness to light. In certain applications, six formulations were more efficient (up to 500%) than commercial Lipofectamine in gene knockdown activity. These formulations had differential internalization by skin cells and the endosomal escape was rapid (minutes range) as shown by the recruitment of galectin 8. The NPs described were effective in the release of siRNA and miRNA but can also be extended to the release of mRNA and other types of RNA. Acute skin wounds treated with the top hit NP complexed with miRNA-150-5p healed faster than wounds treated with scramble miRNA. Thus, light-triggerable NPs offer a new strategy to deliver topically non-coding and coding RNAs.

### TECHNICAL FIELD

This application relates to a light-triggerable nanoparticle library of formulations for controlled release of RNAs.

### BACKGROUND ART

The capacity to regulate intracellular gene expression with RNA-based therapeutics such as small interfering RNAs (siRNAs) or miRNAs has enormous potential for the treatment of many diseases [1-3]. Unfortunately, the intracellular delivery of RNA-based therapeutics is difficult because of their susceptibility to enzymatic degradation and low capacity to cross cell membrane without a vector/carrier.

Several delivery strategies have been developed in the last years for the rapid, to facilitate in vivo translation, and efficient, to escape the endolysosomal compartment, delivery of RNA-based therapeutics based on NPs, scaffolds, nano-needles, among others [1-3]. NPs that either encapsulate or carry RNA-based therapeutics in their surfaces can stabilize the RNA molecules, target potentially specific cell populations and deliver intracellularly the cargo. Unfortunately, NP formulations still offer limited success in terms of endolysosomal escape and temporal delivery of the cargo [4]. For example, in the most efficient formulations, the escape of RNA molecules from the endolysosomal compartment is below 2% [3, 5]. Moreover, with the exception of few cases [6-10], most of the formulations do not allow temporal delivery of the cargo and yet this issue seems very important because for effective knockdown, RNA molecules should be released from the endosomal compartment shortly (≈15 min) [5] after endocytosis.

The hypothesis of the present application is that rapid and efficient delivery systems for RNA intracellular delivery requires the development of NP libraries for the identification of formulations that facilitate cell internalization while enabling temporal control in the delivery of RNA, with potential advantages in terms of endolysosomal escape. Previous studies have used high-throughput screening to identify NPs to release intracellularly non-coding RNAs [11-15]; however, without enabling remote control by an external stimulus such as light. Several strategies have been reported to make light-triggerable NP delivery systems [16]. A frequently used approach is to introduce light cleavable molecules, such as o-nitrobenzyl groups, on the polymer backbone [17]. Light exposure of the NPs leads to the cleavage of the photo-sensitive moiety followed by the disassembly of the formulation.

There are only three studies reporting the use of light-triggerable formulations for the delivery of miRNAs in vivo. In one of the studies, the miRNA was modified with a photolabile caging group sensitive to UV-light [18] and showed limited skin regeneration at macroscopic level. The other studies reported a formulation sensitive to NIR light for the delivery of miRNAs [10, 19]; however, the inorganic nature of the formulation raised some issues for a potential clinical translation.

### SUMMARY

The present application relates to a light-triggerable nanoparticle library of formulations for controlled release of RNAs wherein the formulations comprise:

polymeric nanoparticles comprising photocleavable linker monomers; amine monomers; and bisacrylamide monomers;

wherein the nanoparticles are complexed with RNA;

and wherein the nanoparticles are adapted to be disassembled when exposed to light.

In one embodiment the photocleavable linker is 2-nitro-1,3-phenylene)bis(methylene) diacrylate (P1).

In another embodiment the bisacrylamide monomers are selected from methylenebisacrylamide (A), hexamethylenebisacrylamide (B), cystaminebisacrylamide (C), dihydroxyethylenebisacrylamide (D), bisacryloylpiperazin (E).

In yet another embodiment the amine monomers are selected from ethylenediamine (1), 1,4-diaminobitan (2), 1,6-diaminohexan (3), diethylenetriamine (4), triethylenetetramine (5), pentaethylenehexamine (6), 3,3′-diamino-N-methyldipropylamine (7), 1,2-diaminocyclohexane (8), 1,8-diamino-3,6-dioxoctane (9), 1,13-diamono-4,7,10-trioxatridecane (10), 1,4-bis(aminopropyl)piperazine (11), 1,4-phenylenedimethanamine (12), 1,5-diaminonaphthalene (13), 4,4′-methylenedianiline (14), 1,3-phenylenediamine (15), 1,3-diaminopropane (16), 2,2-dimethyl-1,3-propanediamine (17), 1,3-diamiopentane (18), 2,2′-diamino-N-methyldiethylamine (19), agmatine sulfate (20), 1,4-Bis(aminomethyl)cyclohexane (21), 4,4′-methylenebis(cyclohexylamine) (22), 4,4′-diaminobenzanilide (23), DL-Lysine (24), 3-amino-1-propanol (25), 4-amino-1-butanol (26), 5-amino-1-pentanol (27), 6-amino-1-hexanol (28), 1-(3-aminopropyl)pyrrolidine (29), 1-(3-aminopropyl)imidazole (30), 1-(3-aminopropyl)-4-methylpiperazine (31), histamine (32).

In one embodiment the maximum percentage molar ratio is between 21% and 23% of P1, between 21% and 23% of bisacrylamide and between 54% and 58% of amines.

In another embodiment the molar ratio of P1 per repeating unit of the polymer is 25 (P1):25(bisacrylamide):50(amine).

In another embodiment 90% of the nanoparticles have a size range between 100 and 500 nm.

In another embodiment 20% of the nanoparticles have a zeta potential above 20 mV.

In yet another embodiment 80% of the formulations show 50% count decrease after 10 min of light exposure.

In one embodiment the ratio of siRNA:NP or miRNA:NP is 1:50.

In one embodiment the ratio of mRNA:NP (w/w) varies between 1:5 and 1:100.

In another embodiment it comprises formulations P1A1, P1A7, P1C5, P1C7.

In another embodiment they have a cell transfection time of 10 minutes or less.

In one embodiment the nanoparticles have a complexation efficiency with RNA between 75 and 125%.

The present application also relates to process to produce a light-triggerable nanoparticle library of formulations for controlled release of RNAs as described in any of the previous claims, having the following steps:
Reacting the monomers in a molar ratio of 25 (P1):25(bisacrylamide):50(amine);
End capping the polymers with 20% molar excess of the respective amine 1-32;
Preparing nanoparticles (NPs) by precipitation of the polymers in sterile nuclease free molecular grade water and zinc sulfate;
Complexing of RNAs with the NPs.

The application still related to the use of light-triggerable nanoparticle library of formulations for controlled release of RNAs in skin, eyes and intestines.

### General Description

RNA-based therapies offer a wide range of therapeutic interventions including for the treatment of skin diseases; however, the strategies to deliver efficiently these biomolecules are still limited due to obstacles related to the cellular uptake and cytoplasmic delivery. The present application aims to disclose a light-triggerable polymeric nanoparticle (NP) library composed by several formulations, presenting physico-chemical diversity and differential responsiveness to light, with rapid cell transfection time, 10 minutes or less.

In certain applications, six formulations were more efficient, up to 500%, than commercial Lipofectamine in gene knockdown activity.

These formulations had differential internalization by skin cells and the endosomal escape was rapid, minutes range, as shown by the recruitment of galectin-8. The NPs were shown to be effective in the release of siRNA and miRNA. Acute skin wounds treated with the top hit NP complexed with miRNA-150-5p healed faster than wounds treated with scramble miRNA. Light-triggerable NPs offer a new strategy to deliver topically non-coding RNAs.

In addition to non-coding siRNA and miRNA, these formulations can also be used to release coding mRNA effectively.

The polymeric NPs herein described are produced from photocleavable linker monomers, amine monomers and bisacrylamide monomers.

When exposed to incident light the polymeric nanoparticles disassemble in order to efficiently deliver RNAs to target sites. The disassembly of the nanoparticles occurs due to the presence of a photocleavable linker in the polymeric structure, that is light sensitive.

### BRIEF DESCRIPTION OF DRAWINGS

For easier understanding of this application, figures are attached in the annex that represent the preferred forms of implementation which nevertheless are not intended to limit the technique disclosed herein.

FIG. 1. Light-triggerable NP library and gene knockdown activity. (A) Reaction scheme for the preparation of light-sensitive polymers based in the reaction of bisacrylamides (A-E), photo-cleavable diacrylate (P1) and amines (1-32). (B) Monomers used for the synthesis of the library. (C) Schematic representation of the light disassembly of the NPs, where 1 represents chemical diversity, 2 represents disassembly control and 3 represents differential cell uptake. (D) High throughput screening of NPs for gene knockdown using siRNA. Fold increase of GFP knockdown after 48 h post transfection relative to Lipofectamine (Lipo) for the best 40 formulations. Cells were transfected with the formulations for 10 min and subsequently irradiated for 10 min with a UV lamp. The red bars show GFP knockdown in the best two formulations without UV irradiation. Results are expressed as Mean±SEM (n=3).

FIG. 2. Intracellular trafficking and bioactivity. (A) Galectin-8 recruitment in A7r5-Gal8YFP reporter cells cultured in the presence of P1C7@siRNA-Cy5 or lipofectamine@siRNA-Cy5, as evaluated by confocal microscopy. Cells were transfected with the formulations for 10 min (t=−10 min) and then activated/exposed by UV light (10 min, 365 nm, 1 mW/cm²; t=0 min). White scale bar is 50 μm. (B) Colocalization of Galectin-8 YFP spots with Cy5 (B.1) as well as mean areas of bright Gal8-YFP spots (B.2) and siRNA-Cy5 (B.3). Results are Mean±SEM (n=4-17, 2 technical replicates). (C) Schematic representation of the experimental protocol, where 1 represents formulation, 2 represents wound, 3 represents wash, 4 represents starvation medium, 5 represents light trigger. Confluent human keratinocytes were treated for 4 h with P1C7 NPs or P1C7@miR150 NPs. Lipofectamine complexed with miR150 was used as control. The wound was created by scratching the monolayer of cells. Cell migration was monitored by high-content microscopy. (D.1) Wound closure 48 h post-scratch. Wound area was quantified by ImageJ and normalized to the initial wound area. Results are presented as average ±SEM (n=6-8). Quantification of miR150 (D.2) and cMYB (D.3) gene transcripts by qRT-PCR in keratinocytes treated with P1C7 NPs or P1C7@miR150 NPs, 48 h post wounding. Results are presented as Mean±SEM (n=6-8). Statistical analysis was performed by a student t-test, except for NP P1C7 against time at 0 min which was assessed by one-way ANOVA followed by a Bonferroni post-test. *P<0.05; **P<0.01; ***P<0.001; ****P<0.0001.

FIG. 3. Acute wound healing activity of P1C7@miR150 formulation. (A) Schematic representation of the animal experimental set up, where 1 represents intradermal injection, 2 represents light trigger, 3 represents injection site, 4 represents wound area, 5 represents irradiation area. (B) Wound closure (relative to day 0) in animals treated with PBS, P1C7@scramble or P1C7@miR150. Results are Mean±SEM (n=8). Statistical analyses were performed by student t-test with Welch's correction. #P<0.05, ##P<0.01 between PBS and P1C7-miR150 groups; *P<0.05 between P1C7-scramble and P1C7-miR150 groups. (C) Histological score regarding wound healing. Results are Mean±SEM (n=8). (D) Representative hematoxylin/eosin staining's for wounds at day 10. Scale bar is 500 μm. (E) Expression of cMYB transcripts in skin tissue quantified by qRT-PCR analyses. Data presented as Mean±SEM (n=6-20). **P<0.01.

FIG. 4. Synthesis of a photo-cleavable linker and its incorporation in polymers. (A) Reaction scheme for the synthesis of the photo-cleavable linker P1. (B) 1H spectrum of P1 in CDCl3. (C) 1H spectrum of P1A1 in DMSO-d6 with P1 (x):amine (y):bisacrylamide (z) ratio of 25:50:25. (D) Optimisation of the ratio between P1, bisacrylamide and amine in P1A1 and P1A7 NPs, to obtain NPs that are highly light responsive, and thus photo-cleavable.

FIG. 5. 1H spectra of P1C5 (A) and P1C7 (B) polymers in DMSO-d6 with P1 (x):amine (y):bisacrylamide (z) ratio of 25:50:25.

FIG. 6. Physicochemical properties of the NP library. (A) Scheme illustrating the synthesis of the polymers and formation of NPs, where 1 represents amine, 2 represents bisacrylamide, 3 represents photo-cleavable linker, 4 represents Michael-type addition, 5 represents H2O+ZnSO4, 6 represents centrifugation. (B) NP formation efficiency of the top 50 formulations. NP formation efficiency was calculated by the ratio between the weight of NPs after purification and the theoretical molecular weight of the polymer. (C-D) Frequency distribution of NP diameter (C) and zeta potential (D). (E) Zeta potential of the top 50 positive NPs. The results are expressed as Mean±SEM (n=3). (F) NP count decrease of the top 50 after 10 min UV irradiation (365 nm). The light dissociation of the NPs was calculated by the ratio between the counts (as determined by DLS) before and after light exposure.

FIG. 7. Characterization of P1C7 NPs. (A) Representative image of NPs obtained by TEM. (B) NP diameter distribution as determined by TEM and DLS analyses. For DLS (Brookhaven ZetaPALS), data from five NP (50 μg/mL) samples was collected with five measurement runs (1 min) on each sample. In case of TEM analyses, NP suspensions with 500 μg/mL were applied on carbon coated 200 mesh copper grids, left to air dry and analyzed (FEI-Tecnai Spirit BioTwinG2). Up to 5 images were acquired and analyzed on ImageJ. Results are Mean±SEM (n=3-5, up to 7 technical replicates).

FIG. 8. Determination of the siRNA@NP complex concentration, siRNA:NP ratio, kinetics and effect of light activation/light exposure in gene knockdown. RNAiMAX complexed with siRNA was used as control. Viability and gene knockdown studies was performed in HeLa cells expressing GFP. GFP knockdown was monitored by high content microscopy. (A) HeLa cell viability after transfection with NPs (siRNA:NP ratio=1:50) at different concentrations for 4 h. Cell nuclei were stained with Hoechst H33342 and propidium iodide after 48 h. Cell viability was calculated as the % of dead nuclei from the total count of nuclei. (B) Effect of siRNA:NP (w/w) ratio keeping NPs concentration constant at 20 μg/mL in terms of GFP knockdown analysed after 48 h. (C) Effect of transfection time of NP@siRNA complexes (siRNA:NP ratio=1:50; 20 μg/mL) in GFP knockdown analysed after 48 h. (D) Influence of UV light irradiation (365 nm, 1 mW/cm2) on siRNA@NP complex and

consequently, GFP knockdown. Cell transfection was performed for 10 min followed by 10 min of UV irradiation. GFP knockdown was analysed at 48 h. Results are Mean±SEM (n=4). Statistical analysis was performed using an unpaired student t-test; **P<0.005.

FIG. 9. Complexation capacity of the NPs for siRNA as well as cytotoxicity and cellular internalisation of NP@siRNA complexes. (A) Schematic representation of siRNA complexation with NPs (siRNA:NP ratio=1:50) and cellular internalisation studies of NP@siRNA complexes (20 μg/mL) with transfection time of 10 min, where 1 represents siRNA-Cy5, 2 represents centrifugation, 3 represents siRNA complexation efficiency, 4 represents cell transfection. (B) siRNA complexation efficiency of the top 50 formulations determined in NPs@siRNA-Cy5. (C) HeLa cell viability at 48 h post transfection without UV irradiation. Cell nuclei were stained with Hoechst H33342 and propidium iodide at 48 h, and cell viability calculated as the % of dead nuclei from the total count of nuclei. (D) Percentage of cells stained for NPs@siRNA-Cy5 at 48 h post-transfection. The top 50 conditions with higher NP internalisation in HeLa cells are displayed. Results are Mean±SEM (n=3).

FIG. 10. Schematic representation of image analysis. (A) Image analysis steps: (1) definition of healthy cell population to analyse; (2) definition of area to measure GFP levels and (3) creation of cytoplasm mask by subtracting the nuclear mask from the cell mask, where 4 represents cells, 5 represents viable cells, 6 represents nuclear mask, 7 represents cell mask, 8 represents cytoplasm mask, 9 represents apoptotic cell (small and intense nuclei), 10 represents dead cell (PI cells). (B) Image analysis was conducted using In Cell Developer Software which implements machine learning techniques. Cell viability was analyzed calculating the percentage of nuclei with form factor >0.95 and at least 10% overlap with propidium iodide staining, in the total nucleus population. By subtraction of the dead masked nuclei from the total nuclei population, a healthy nucleus mask can be defined. Consecutively the healthy nucleus mask was dilated to cells and the nucleus mask was subtracted to achieve a cytoplasm mask, where GFP fluorescence can be measured minimizing artefacts from other stains (i.e. H33342) and flattening the detection pane. GFP knockdown can then be calculated as percentage decrease of GFP fluorescence signal relative to 0% GFP knockdown control from untreated HeLa-GFP cells and 100% GFP knockdown of HeLa background control cells.

FIG. 11. Internalization mechanism of P1C7@siRNA-Cy5 NPs. (A) Schematic representation of the experimental protocol, where 1 represents seed cells prior to experiment, 2 represents preincubation 30 minutes with chemical inhibitors, 3 represents incubation with P1C7@siRNA-Cy5 for 1 hr, 4 represents trypsinize and wash 3× with PBS, 5 represents FACS. (B) Effect of temperature in the cellular uptake of P1C7@siRNA-Cy5 NPs. (C) Uptake of P1C7@siRNA-Cy5 in the presence of several endocytosis inhibitors: filipin III inhibits cholesterol dependent internalization mechanisms, nocodazole inhibits microtubule dependent pathways, polyinosinic acid inhibits scavenger receptors, dansylcadaverine and dynasore inhibits clathrin-mediated endocytosis, cytochalasin D inhibits all pathways dependent on actin (including macropinocytosis) and ethylisopropylamiloride (EIPA) inhibits macropinocytosis. The concentrations tested for each inhibitor were confirmed before the experiment to be noncytotoxic. Cellular internalisation of P1C7@siRNA-Cy5 NPs was evaluated by flow cytometry. Results are expressed as Mean±SEM (n=3).

FIG. 12. NP internalisation in human skin cells. Fibroblasts (A), keratinocytes (B) and endothelial cells (C) were transfected with NP@siRNA-Cy5 formulations (20 μg/mL) for 1 h, washed to remove the non-internalised NPs and finally stained (CFSE for cell membrane; Lysotracker red for endolysosome; H33342 for cell nuclei) for confocal microscopy examination. Formulations with high (P1C5 and P1C7) and low (P1D30) gene knockdown efficiency as well as lipofectamine (Lipo) were evaluated for internalisation studies. Cell internalisation was monitored by the colocalization of siRNA-Cy5 with Lysotracker red. Results are presented as Mean±SEM (n=2 independent samples, 3-9 microscope fields). Statistical analyses were performed by one-way ANOVA followed by a Bonferroni multi-comparison test (*P<0.05; **P<0.01; ***P<0.001; ****P<0.0001). (D) Representative confocal microscopy images showing the colocalization of P1C7@siRNA-Cy5 formulation with endolysosomal compartment (Lysotracker red) for 3 cell types. Scale bar is 50 μm.

FIG. 13. In vitro wound healing activity of P1C7@miR150 NPs. Confluent human keratinocytes were treated for 4 h with P1C7@miR150 without light activation/exposure or P1C7@miR150 with light activation/exposure. Lipofectamine complexed with miR150 (Lipo@miR150) was used as control. The wound was created by scratching the monolayer of cells. Cell migration was monitored by high-content microscopy. (A) Representative images of the wound healing process at 48 h post wounding. Scale is 0.05 cm.

FIG. 14. Photo-disassembly of NPs through a skin barrier. (A) Scheme of the methodology used. A 2 cm×2 cm skin (thickness of 260-290 μm as measured by a caliper) was placed in a plastic petri dish on top of a thermal power sensor (Thorlabs s310c). The tissue was then irradiated with a 405 nm laser at 80 mW/cm² during 1 min. Laser attenuation values were calculated by normalising against laser power values obtained with the empty petri dish. (B) Blue laser attenuation. Results are expressed as Mean±SEM (n=3). (C) Blue laser (405 nm, 80 mW, 5 min) disassembly of P1C7 NPs placed below skin. Results are presented as Mean±SEM (n=3).

FIG. 15. Wound healing activity of P1C7@miR150 formulations. Representative images of the wound healing process immediately after the surgery and at days 3, 5 and 10.

FIG. 16. Table 1: Information about the chemical name, CAS and vendor of the monomers used to generate the NP library.

FIG. 17. Table 2: Polymer molecular weight change of purified P1C5 and P1C7 in DMSO (0.8 M) upon irradiation with a UV lamp at 365 nm for 10 min and summary of the NPs properties.

FIG. 18. Sequence of primers used in qRT-PCR experiments

### DETAILED DESCRIPTION

Now, preferred embodiments of the present application will be described in detail with reference to the annexed drawings. However, they are not intended to limit the scope of this application.

A light-triggerable NP library for the intracellular delivery of RNA-based therapeutics was prepared and characterized. The preparation of such NP library required the use of simple synthetic schemes to be implemented in a high-throughput way, avoiding long purification steps. Thus, Michael-type addition chemistry was selected to produce polymers with chemical diversity [5, 11, 13].

A photo-cleavable linker was synthetized, based on o-nitrobenzyl chemistry, which was reacted with a set of bisacrylamide and amine monomers. The synthesized polymers were precipitated in water to form NPs and then can be complexed with non-coding or coding RNAs (in one embodiment siRNA or miRNAs).

The polymeric NPs herein described are produced from photocleavable linker monomers, amine monomers and bisacrylamide monomers.

In one embodiment the photocleavable linker is 2-nitro-1,3-phenylene)bis(methylene) diacrylate (P1).

In one embodiment, the disclosed NPs can also be complexed with mRNA in a molar ratio mRNA:NPs between 1:50 to 1:100.

In one embodiment, the molar ratio of miRNA:NPs is 1:50.

In one embodiment the molar ratio of P1 per repeating unit of the polymer is 25 (P1):25(bisacrylamide):50(amine).

In one embodiment the maximum percentage molar ratio is between 21% and 23% of P1, between 21% and 23% of bisacrylamide and between 54% and 58% of amines.

In one embodiment the polymeric nanoparticles are disassembled when exposed to light. In one embodiment the light ranges from ultra-violet to infrared (IR) wavelengths. In the case of infrared light, pulsed IR light in short periods of time are able to also disassemble the NPs.

In one embodiment the nanoparticles have a complexation efficiency with RNA between 75 and 125%.

The bisacrylamide monomers are selected from methylenebisacrylamide (A), hexamethylenebisacrylamide (B), cystaminebisacrylamide (C), dihydroxyethylenebisacrylamide (D), bisacryloylpiperazin (E).

The amine monomers are selected from ethylenediamine (1), 1,4-diaminobitan (2), 1,6-diaminohexan (3), diethylenetriamine (4), triethylenetetramine (5), pentaethylenehexamine (6), 3,3′-diamino-N-methyldipropylamine (7), 1,2-diaminocyclohexane (8), 1,8-diamino-3,6-dioxoctane (9), 1,13-diamono-4,7,10-trioxatridecane (10), 1,4-bis(aminopropyl)piperazine (11), 1,4-phenylenedimethanamine (12), 1,5-diaminonaphthalene (13), 4,4′-methylenedianiline (14), 1,3-phenylenediamine (15), 1,3-diaminopropane (16), 2,2-dimethyl-1,3-propanediamine (17), 1,3-diamiopentane (18), 2,2′-diamino-N-methyldiethylamine (19), agmatine sulfate (20), 1,4-Bis(aminomethyl)cyclohexane (21), 4,4′-methylenebis(cyclohexylamine) (22), 4,4′-diaminobenzanilide (23), DL-Lysine (24), 3-amino-1-propanol (25), 4-amino-1-butanol (26), 5-amino-1-pentanol (27), 6-amino-1-hexanol (28), 1-(3-aminopropyl)pyrrolidine (29), 1-(3-aminopropyl)imidazole (30), 1-(3-aminopropyl)-4-methylpiperazine (31), histamine (32).

The NPs were characterized for their size, zeta potential, light disassembly properties, cellular internalization and gene knockdown activity. The formulations with high activity were characterized for their endolysosomal escape.

For proof of concept, the efficacy of the formulation in a wound healing animal model using miRNA-150-5p was demonstrated, recently identified to be important in wound healing.

Biodegradable NP formulations were identified that are able to transfect efficiently in vivo miRNAs, siRNA or mRNA, and enhance significantly wound healing kinetics.

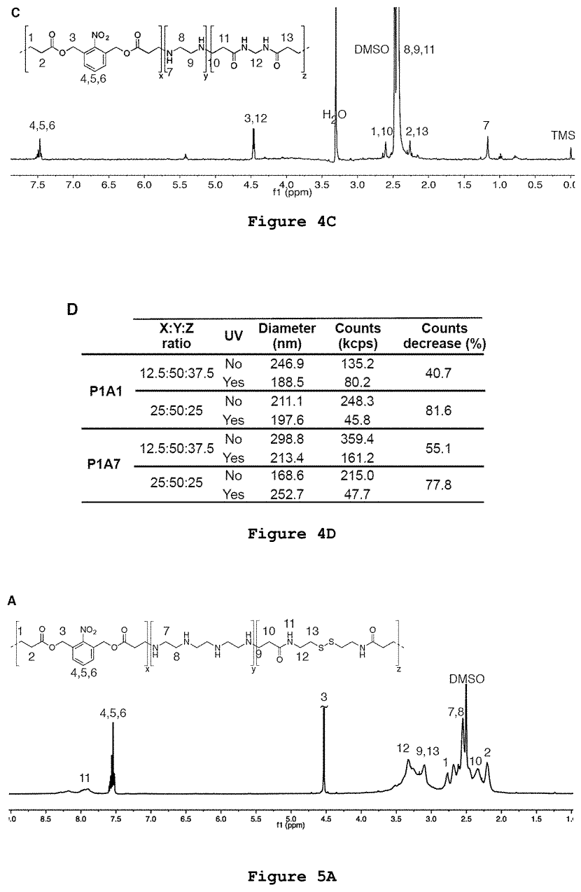

To confer the light sensitivity to the polymers, a photo-cleavable linker P1 was introduced in the polymer backbone (FIGS. 4A and 4B).

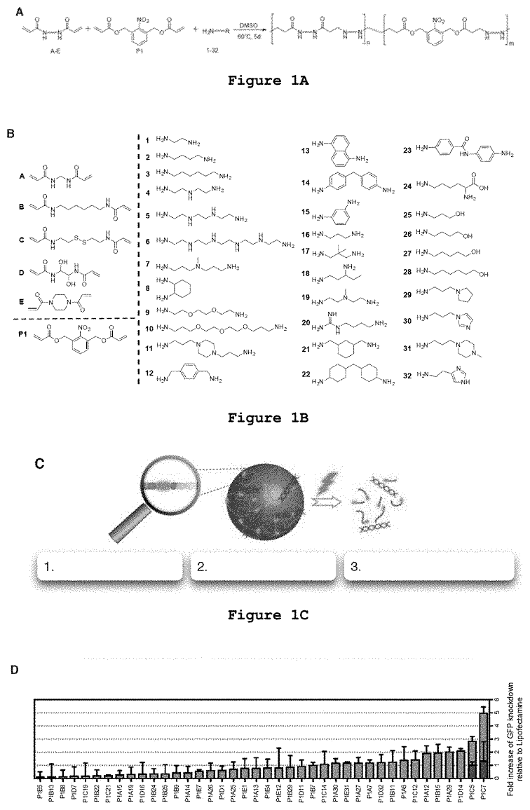

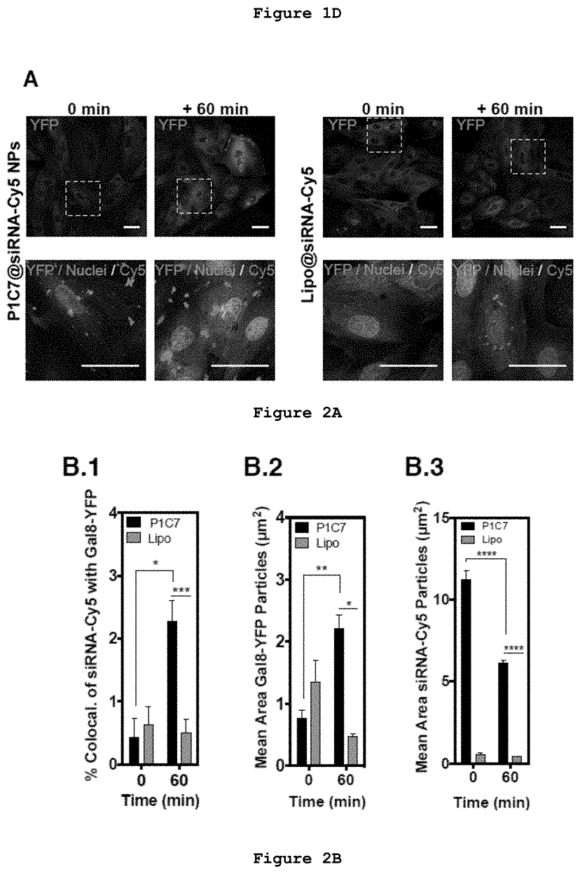

The library was prepared by the addition of monomers (P1, amine and bisacrylamide monomers) in dimethyl sulfoxide (DMSO), for 5 days, at 60° C. (FIGS. 1A-1C). To confer high light sensitivity to the synthesized polymers and consequently NPs, the ratio of P1 per repeating unit of the polymer was 25 (P1):25 (bisacrylamide):50 (amine) (FIGS. 4C and 4D).

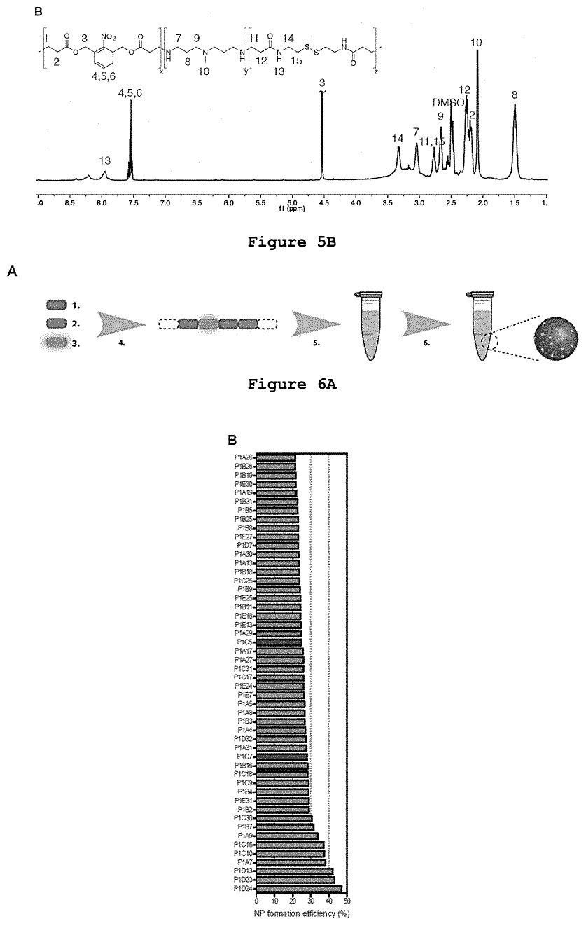

At the end of the reaction, the unreacted acrylamide groups were capped with amines since previous studies have demonstrated that amine-terminated polymers had higher transfection efficiencies [20]. The efficiency of the capping procedure was high as no measurable acrylate proton signals (5-7 ppm) were observed by 1H-NMR (FIG. 4C and FIG. 5).

The synthesized polymer library had (i) a large variety of side groups (ii) disulfide bonds that were relative stable in physiological conditions, pH 7.4, but were rapidly degraded in intracellular reductive environments and (iii) different solubility and hydrophilicity in aqueous solution.

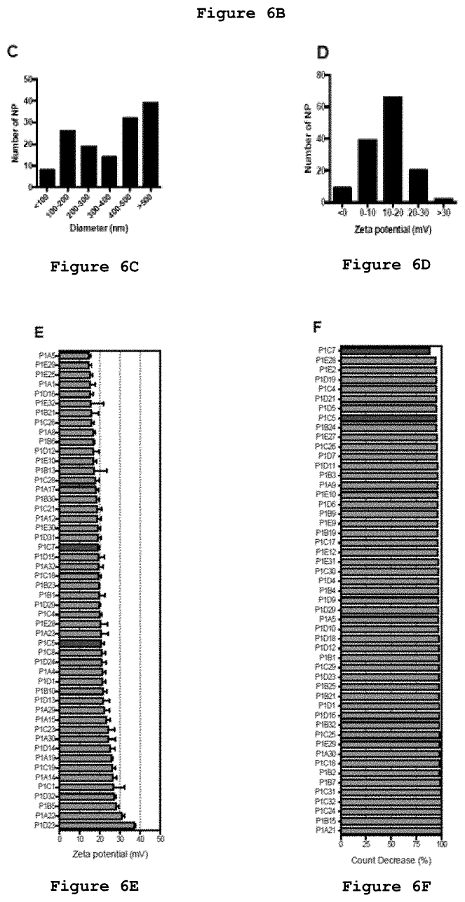

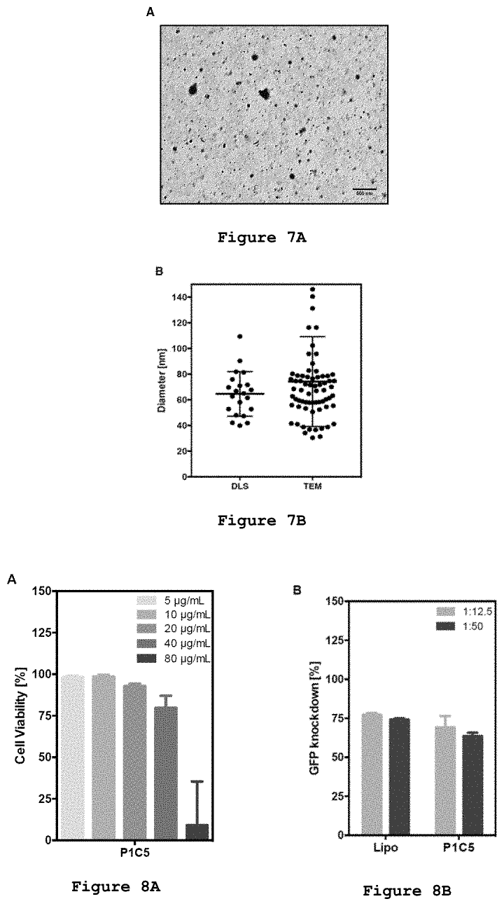

The library of polymers was then precipitated in water to form NPs (FIG. 6A) with a 90% efficiency in NP formation. The average yield of NP formation was 20.7±15.3%, having 10% of the polymers yields above 25% (FIG. 6B). 90% of the NPs had a size range between 100 and 500 nm (FIG. 6C) and 20% of the NPs a zeta potential above 20 mV (FIGS. 6D-6E), as evaluated by dynamic light scattering (DLS). TEM analyses confirmed the size range obtained by DLS (FIG. 7). The light responsiveness of the NPs was then evaluated (FIG. 6F), by the quantification of number of counts per second before and after light exposure, as assessed by DLS. At least 80% of the formulations showed 50% count decrease after 10 min of UV light exposure. Light sensitivity seemed to be independent on the characteristics of the bisacrylamide monomers.

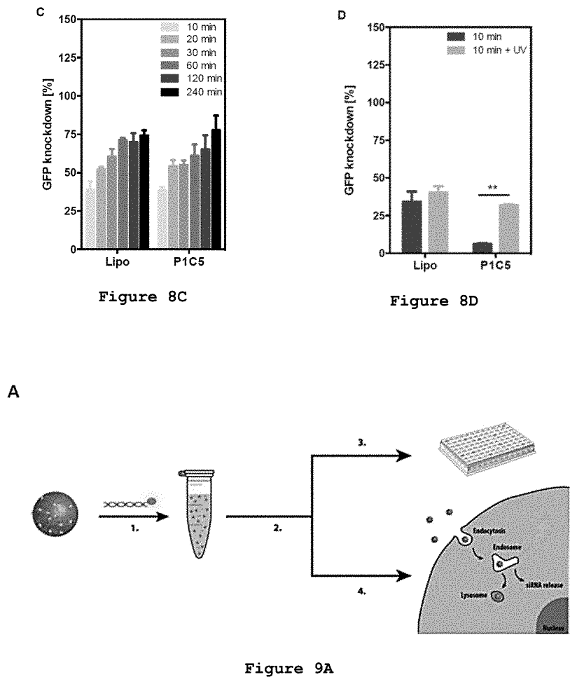

To form the NP@siRNA complexes, both NP and siRNA were mixed to promote electrostatic interactions. Based on preliminary tests with P1C5 formulation, a ratio siRNA (against GFP):NP (w/w) of 1:50, a concentration of NP@siRNA of 20 μg/mL and a transfection time of 10 min were selected for running the NP library (FIG. 8).

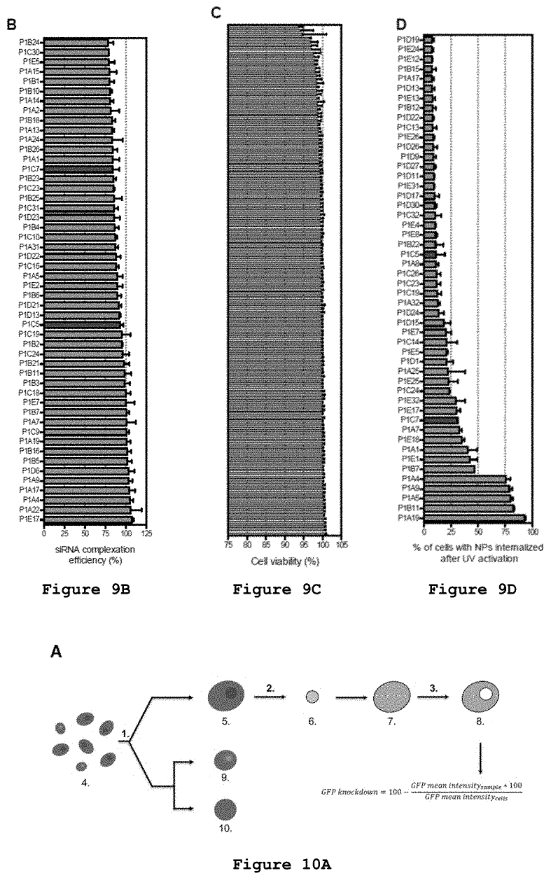

For in vivo applications (see below), it is desirable that the formulations are rapidly internalized by cells to reduce their washing from the place they are administered and thus a 10 min-transfection time was used for subsequent studies. To run the library, the NPs were complexed with siRNA labeled with a Cy5 tag, followed by the centrifugation of the NPs, the quantification of the concentration of siRNA not immobilized onto the NPs in the supernatant, and the use of NP@siRNA complexes for cell transfection (FIG. 9A). In average, 79.0±13.5% of the initial siRNA was immobilized onto the NPs. Thirty two percent of the formulations did immobilize more than 80% of the initial siRNA (FIG. 9B).

In one embodiment, the formulations more effective binding siRNA were those NPs formed by bisacrylamide A, C and E and diamines 2, 3, 4, 5, 6, 7, 11, 16, 21 or 22. The results suggested that the binding of siRNA was not only dependent on positive zeta potential but also on the presence of aliphatic moieties in the polymer backbone.

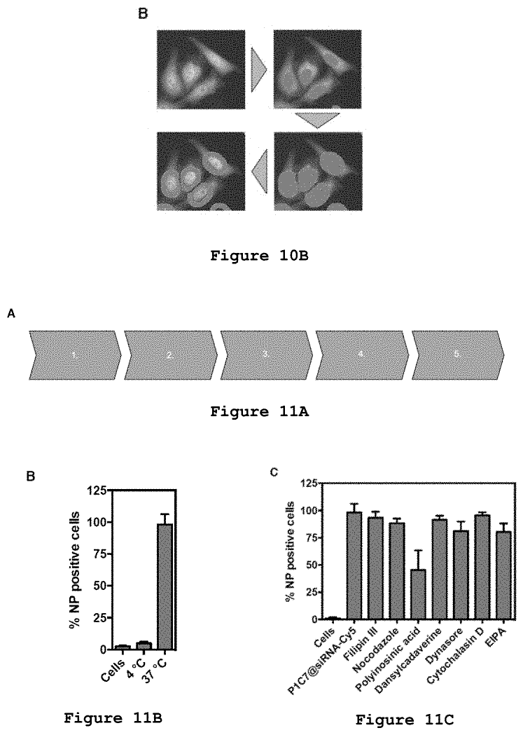

To evaluate the knockdown properties of the NP@siRNA complexes, HeLa cells stably expressing eGFP were transfected with the formulations. Cells were transfected with NP@siRNA-Cy5 (siRNA:NP 1:50; 20 μg/mL) complexes for 10 min, washed to remove non-internalized complexes, either irradiated or not for 10 min and cultured for additional 48 h. Lipofectamine was used as control. High-content imaging was used to monitor simultaneous several parameters in the same screening experiment, such as cell viability, NP internalization (from the Cy5 tag of the siRNA) and GFP knockdown (FIG. 10). For the concentration tested, the formulations had no significant impact in cell viability (cell viability >90%) (FIG. 9C) but they showed significant differences in the delivery of siRNA within cells (FIG. 9D).

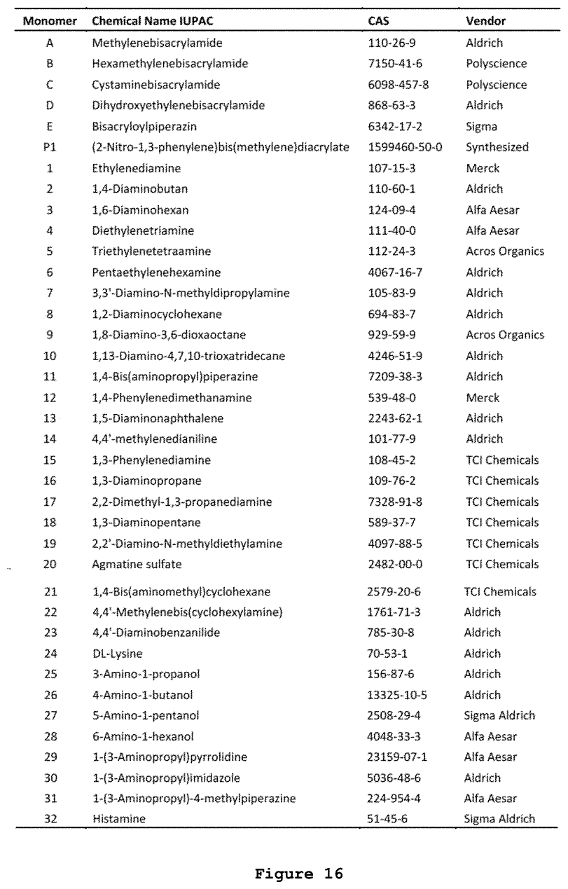

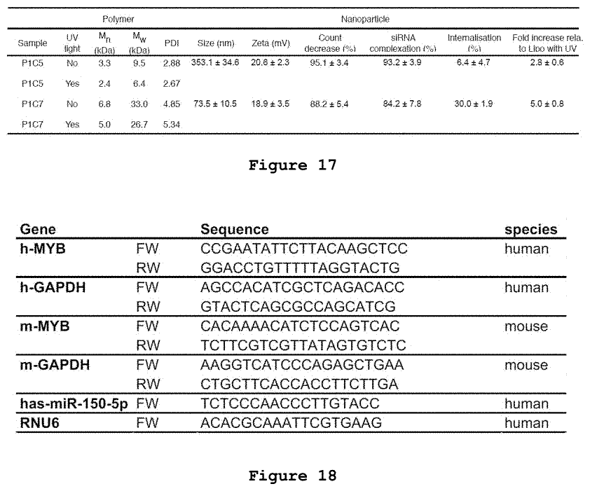

Importantly, in some experiments, six formulations were more efficient than commercial Lipofectamine to knockdown GFP (FIG. 1D). These formulations contained bisacrylamide A (shortest aliphatic), C (bioreducible disulphide bond) and diamines 1 (short aliphatic), 4-7 (increased nitrogen content) and 10-11 (ethyleneglycol containing units) (FIG. 16). Two formulations (P1C7 and P1C5) were selected for further characterization due to their high ability to knock down the reporter cell line. Importantly, the knock down activity of the formulations was superior after light activation, i.e. exposure to light to promote disassembly, as compared to the same formulations without light activation/exposure, highlighting the temporal control of their biological activity. P1C7 formulation was the most promising one because of its efficiency and temporal control of GFP knockdown (FIG. 17). The internalization of P1C7@siRNA-Cy5 NPs in HeLa cells (was endocytic and mainly mediated by scavenger receptors (FIG. 11).

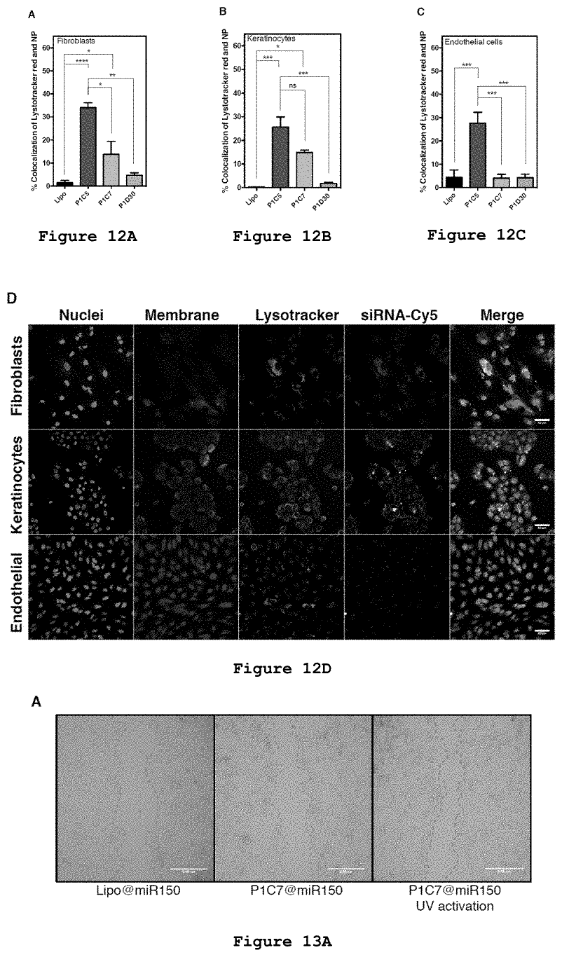

Light-triggerable NPs might have potential use in skin applications [10, 19]. Therefore, it was evaluated whether some of formulations had different tropism to skin cells. For this purpose, the internalization of two formulations with high GFP knockdown, P1C7 and P1C5, as well as one formulation with low GFP knockdown (P1D30) were tested against human skin cells, specifically, fibroblasts, keratinocytes and endothelial cells (Ecs). Cells were transfected with NPs@siRNA-Cy5 or lipofectamine@siRNA-Cy5 (FIG. 12). The results showed significant differences in NP internalization according to each cell type demonstrating that cell internalization was dependent in the chemistry of the formulation. P1C7 formulation had higher tropism to fibroblasts and keratinocytes than ECs. Based in the knockdown efficiency and skin cell internalization pattern, P1C7 NPs were selected for subsequent studies.

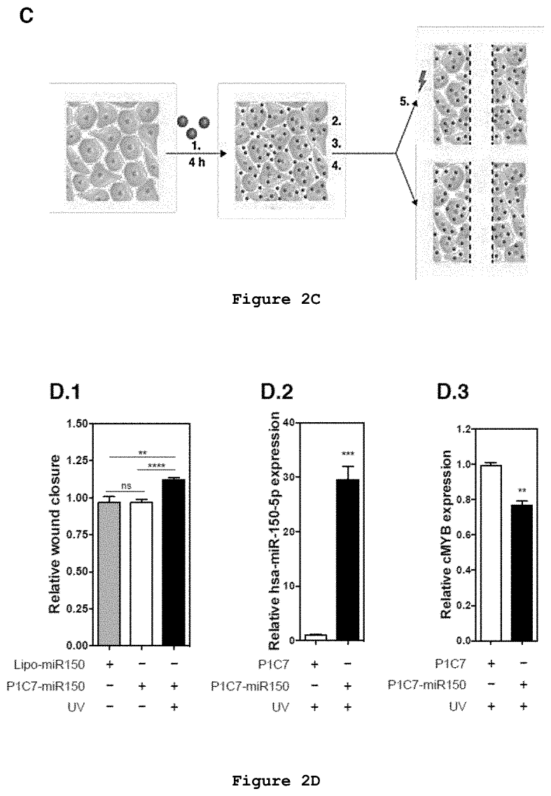

Previous studies have demonstrated that efficacy of RNA silencing mechanism is directly correlated with endosomal escape [3, 11], which is mediated by the recruitment of galectin-8 to the RNA releasing endosomes [5]. To evaluate whether efficacy of P1C7 formulation in RNA silencing was connected to the rapid endosomal escape, the kinetics of galectin-8 recruitment was measured on A7r5-Gal8YFP reporter cells [21]. Cells were transfected with P1C7@siRNA-Cy5 NPs for 10 min, after which, they were exposed to UV light for 10 min and then incubated for 60 min. Lipofectamine complexed with the same amount of siRNA-Cy5 was used as control. The area of siRNA-Cy5 NPs decreased overtime indicating the disassembly of the NPs within cells. Cells transfected with P1C7@siRNA-Cy5 showed higher number of foci with galectin 8 than the ones transfected with lipofectamine, and the foci number as well as the foci area increased overtime (FIGS. 2A and B). These results indicate that P1C7 formulation delivered more efficiently the siRNA to the cell cytoplasm than lipofectamine.

To extend the application of P1C7 NPs, the formulation was used for the delivery of miRNAs. NPs were complexed with miRNA-150-5p (from now on termed as miRNA150), a miRNA that has been recently identified by us to have relevance in human keratinocyte migration (data not shown). Cells were transfected with P1C7 NPs, P1C7 NPs@miRNA150 or lipofectamine@miRNA150 (FIG. 2C). Cells were then wounded, and cell migration was monitored. At 48 h post wounding, cells transfected with non-irradiated P1C7 NPs@miRNA150 showed increased migration as compared to NPs without miRNA150 (FIG. 2D.1 and FIG. 13). Importantly, cells transfected with P1C7 NPs@miRNA150 and activated by light to promote disassembly of the NPs, showed increased migration as compared to cells transfected with non-activated NPs or lipofectamine, both complexed with miRNA150. As expected, the increased migration of cells seems to be mediated by an increase in the intracellular levels of miRNA150 (FIG. 2D.2) and by the knock down of cMYB gene (FIG. 2D.3), a direct target of the miRNA [22].

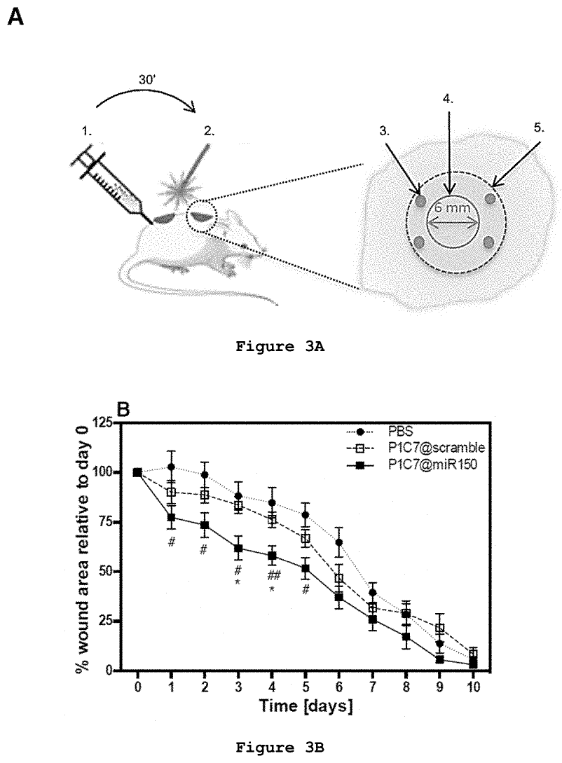

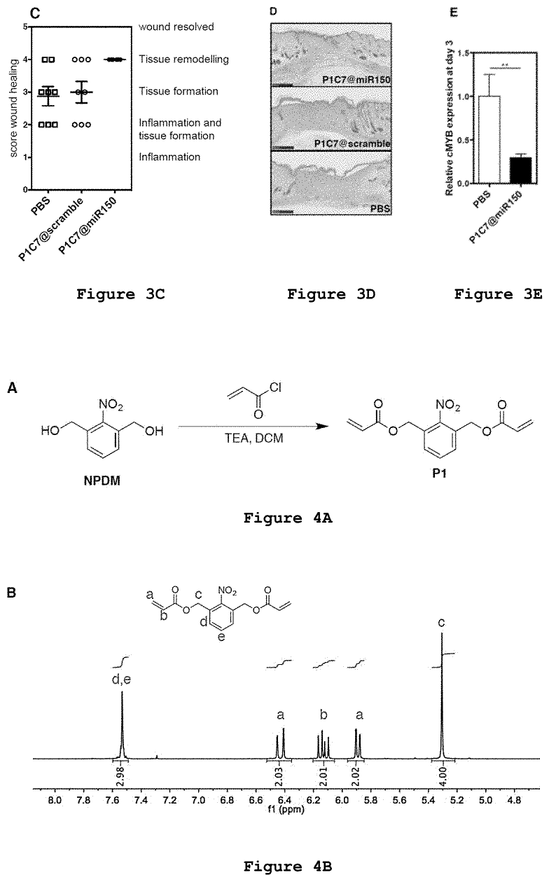

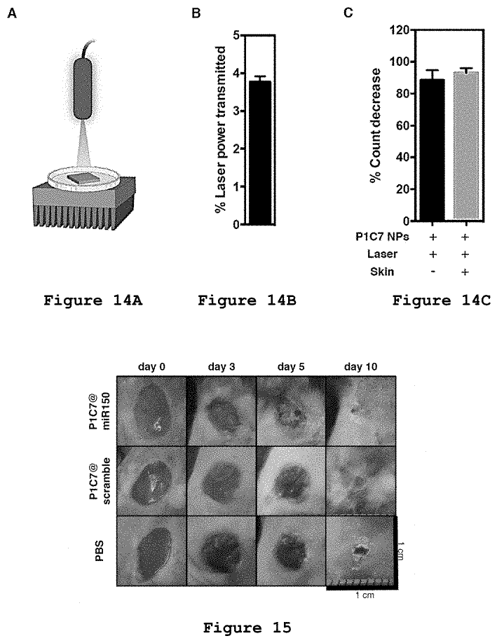

Next, it was evaluated whether P1C7@miRNA150 NPs could function in vivo, in an acute wound healing animal model. Initially, it was investigated whether the formulation could be activated in vivo by a blue laser after subcutaneous transplantation (FIG. 14A). The results showed high NP disassembly even if the attenuation of the laser was significant, only 4% of the laser was able to cross the skin barrier of 300-400 μm, (FIGS. 14B and 14C). Then, P1C7 NPs@miRNA150 was administered subcutaneously in the borders of wounds and allowed the NPs to be internalized by the skin tissue for 30 min, followed by their activation to promote disassembly by a blue laser for 5 min (FIG. 3A). P1C7 NPs@scramble miRNA or vehicle (PBS) were used as controls. Wounds treated with light triggered P1C7@miRNA150 NPs healed faster as compared to wounds treated with PBS or P1C7 NPs@scramble miRNA, being statistically significant for some of the days (FIG. 3B). These results were also confirmed by histological analyses showing qualitative differences in the wound healing process and re-epithelization (FIGS. 3C and 3D). Wounds treated with PBS were in stages of inflammation and tissue formation until tissue remodeling, whereas wounds treated with P1C7 NPs@miRNA150 were all in the tissue remodeling phase (FIG. 3D).

To show that indeed the regenerative program was mediated by miRNA150, the expression of cMYB gene, a direct target of miRNA150, was evaluated at day 3 by qRT-PCR (FIG. 3E). The percentage of cMYB gene transcripts were 60% lower in wounds treated with P1C7@miRNA150 NPs than with P1C7 NPs.

In conclusion, a light-triggerable NP library of formulations was successfully synthesized for efficient in vivo small non-coding RNA delivery or coding mRNA.

In some applications, the library screening revealed six formulations which were more efficient in cell transfection and RNA silencing as the commercial agent Lipofectamine with additional temporal control over the release of the small non-coding RNA. P1C7, as leading formulation, was shown to be a rapid transfection agent with fast endosomal escape and high efficiency not only for siRNA mediated gene silencing but also for microRNAs. Moreover, it was demonstrated high efficacy and significance of light-triggered microRNA delivery in a wound healing animal model. Taken together, light-triggerable NP formulations give an extra level of control in the delivery of non-coding or coding RNAs which may enhance their bioactivity.

The application of the described library of formulations is the delivery of non-coding or coding RNAs in exposed parts of the body, such as skin and eyes, where light can reach easily and activate the disassembly of the NP formulations, or in the intestines where light can be used in combination with laparoscopy techniques.

Synthesis of the Photocleavable Linker P1

The photocleavable linker P1 (2-nitro-1,3-phenylene)bis(methylene) diacrylate was synthesized and purified according to a previously reported procedure (Biomaterials 2014, 35, 5006-5015). Briefly, to a solution of (2-nitro-1,3-phenylene)dimethanol (5.5 g, 30 mmol, ARCH Bioscience) in anhydrous dichloromethane (38 mL, Fisher Chemical), was added dropwise trimethylamine (10.5 mL, 75 mmol, Sigma-Aldrich) under argon atmosphere. Then, acryloyl chloride (9.8 mL, 120 mmol, Merck) was added dropwise into the reaction mixture using a dropping funnel over 15 min at 0° C. The mixture was stirred for additional 18 h at room temperature (20-30° C.). After removing the formed solid by filtration, the filtrate was dried under vacuum and then resuspended in ethyl acetate (Fisher Chemical). The resulting solution was washed with saturated sodium chloride solution and then dried overnight with sodium sulfate. The final product was obtained after purification by silica gel chromatography (hexane:ethyl acetate as eluent, 1:1, v/v) as a white crystal.

Optimization of the Photocleavable Linker Ratio in Poly(Amido Amine)s

To optimize the amount of the photocleavable linker in the poly(amido amine), ratios of 25:75 and 50:50 of P1 to A (methylenebisacrylamide) were used in the synthesis of some polymers. To calculate the ratio of incorporation of P1 into the polymer, polymers were precipitated in water, lyophilized, resuspended in DMSO-d6 and analyzed by 1H-NMR (Bruker Avance III 400 MHz) relative to TMS. Size and count decrease of the nanoparticles after UV exposure were measured by DLS.

Optimization of the Ratio siRNA:NP, Transfection Time and Irradiation Time for Efficient Gene Knockdown

Several parameters were optimized for the high throughput screening of NP@siRNA mediated gene silencing capacity of the polymeric NP library. Firstly, siRNA:NP ratio was optimized to maximize GFP knockdown. Therefore, NPs (200 μg/mL) were complexed with siRNA against GFP in ratios of 1:12.5 and 1:50 for 2 h in nuclease free sterile water shaking on an orbital shaker (250 rpm) at room temperature (20-30° C.). To evaluate bioactivity of the complexes, HeLa-GFP cells were seeded at a density of 40.000 cells/mL for 24 h prior the experiment. Cells were transfected for 4 h with NP@siRNA complexes (20 μg/mL) in starvation (DMEM). Cells were then washed, fresh medium with reduced serum (DMEM, 5% FBS, 0.5% PenStrep) added and cells were cultured for 48 h. At the end, cells were stained with H33342 and PI (both 0.25 μg/mL) and analyzed by fluorescence microscopy on a high-content microscope (In Cell Analyzer 2200). Cell viability and GFP knockdown were quantified as described in high content imaging section below.

In a separate experiment, the transfection time was optimized. The motivation here was to identify a time relatively short that could lead to significant gene knockdown. Cells were transfected with NP@siRNA complexes (20 μg/mL; 1:50 siRNA:NP) from 10 min to 4 h. In a separate experiment, different UV light sources were evaluated for NP disasembly within cells. Cells were placed (right after transfection and medium replacement) on a 20 cm support (distance from top 15 cm) in a transilluminator (UVP BioSpectrum 500) and irradiated with 365 nm light (1 mW/cm²) from the top for 10 min. In both experiments cells were cultured in medium with reduced serum (DMEM, 5% FBS, 0.5% PenStrep) until 48 h. Cells were stained with H33342 and PI (both 0.25 μg/mL) and analyzed by fluorescence microscopy on a high-content microscope (In Cell Analyzer 2200) for GFP knockdown (described in high content imaging section below).

Synthesis of Polymers with Photocleavable Moieties

Prior to synthesis, diamines (1-32), bisacrylamides (A-E) and photocleavable linker P1, were diluted to 1.6 M in DMSO each. Specifications of all monomers can be found in FIG. 16, Table 1. The monomers (25 μL of P1, 25 μL of bisacrylamides A-E, 50 μL of amines 1-32) were added to a polypropylene 96 well plate, the plate sealed with aluminum foil and then incubated at 60° C. under agitation (orbital shaker, 250 rpm) for 5 days. Polymer synthesis was performed at monomer concentration of 0.8 M at the start of the reaction. Polymers were finally end capped with 20% molar excess (10 μL to 100 μL reaction volume) of the respective diamine 1-32 for 2 h (60° C., 250 rpm) and stored at 4° C. until usage.

Gel Permeation Chromatography (GPC) Analyses

Number average molecular weights (Mn) and molecular weight distributions (Mw/Mn) were measured by GPC on a HPLC Agilent 1260 system equipped with a guard column (Agilent, Aquagel, 10 mm, 10 μm) followed by three columns: (i) Agilent, Aquagel-OH 40, 300×7.5 mm, 8 μm, (ii) Agilent, Aquagel-OH 50, 300×7.5 mm, 8 μm and (iii) Phenomenex, Polysep-GFC-P2000, 300×7.8 mm, range 100-10 k Da, connected to a UV (254 and 280 nm) and RI detector (Agilent). An acetate buffer (0.5 mol/L, pH=4.5) was used as an eluent, at a flow rate of 0.7 mL/min and 35° C. Polyethylene oxide standards (EasyVial PEG/PEO, range 194-1000 k Da) were used to calibrate the SEC, since it has been demonstrated that such eluent composition allows PEO to be a suitable calibration standard for poly(amido-amines)1.

NP Preparation and Activation (i.e. Exposure to Light to Promote Dissasembly)

For the high-throughput screening of NPs, NPs were prepared in sterile conditions using sterilized 96-deepwell polypropylene plates (VWR). Therefore, each polymer solution (15 μL; in DMSO) was precipitated into sterile nuclease free molecular grade water (960 μL) with subsequent addition of sterile zinc sulfate (25 μL, 1M). Plates were sealed with PP adhesive seals and incubated shaking (250 rpm) on an orbital shaker at room temperature (20-30° C.) overnight. NPs were purified by centrifugation at 4° C., 8000 g for 8 min. The mass concentration of each purified formulation was determined after lyophilizing samples. The efficiency of NP formation was up to 47%, calculated according to equation:

$$NP formation efficiency ( \% ) = \frac{{M}_{NP}}{{M}_{polymer}} \times 100$$

where MNP denotes the weight of material recovered after NPs purification and freeze-drying and M polymer is the theoretical polymer weight.

NP size and zeta potential analyses. The size and zeta potential of NPs was measured by a ZetaPALS analyzer (Brookhaven Instruments Corp.). NPs were resuspended in 1 mM KCl and diluted to achieve average count rates about 200 kcps to perform the DLS measurement. Values were expressed as the mean of 5 measurement runs, each with a duration of 1 min. To determine light sensitivity of the NPs, a duplicate of the sample was used. NP disassembly was triggered by a UV lamp (365 nm, 100 Watt, 5 cm distance, 10 min). Samples NP size and average count rates were determined as for the non-irradiated sample. Light sensitivity is expressed as percent count decrease respective to the initial average count rate, which is an indicator for NP concentration.

TEM Analyses

A suspension of P1C7 NPs (500 μg/mL) was prepared in molecular grade water. A droplet of each solution was added on the surface of carbon coated 200 mesh copper grid and left air-dry for 5 h at room temperature (20-30° C.) in a closed petri dish. NPs were viewed with a FEI-Tecnai Spirit BioTwinG2 electron microscope. Digital images were acquired with coupled side mounted CCD camera MegaView III-SIS. The diameter of NPs was analysed with the Particle Tool from ImageJ.

High-Throughput Complexation of NPs with siRNAs

NPs were suspended in sterile nuclease free molecular grade water to a concentration of 400 μg/mL. Complexation with siRNA against eGFP (GFP Duplex I, GE Dharmacon) was then done at a ratio of 1:50 (siRNA:NP, w/w) in a 96-deepwell polypropylene plate (VWR). From each NP formulation, an aliquot (50 μL) was added to a deep well plate, following the plate layout for subsequent cell transfection. A solution of siRNA containing 4 μg/mL siRNA and 4 μg/mL Cy5-tagged siRNA was prepared in sterile molecular grade, nuclease free water. The siRNA solution (50 μL) was added to the NPs in the same volume (50 μL) using a multichannel pipette. As control for siRNA activity and transfection, the same procedure was followed for lipofectamine RNAiMAX (15 μl/mL; Invitrogen). The plates were sealed with adhesive polypropylene seals and allowed to incubate shaking at room temperature (20-30° C.) for 2 h on an orbital shaker (250 rpm, room temperature (20-30° C.)). Samples were then diluted 1:10 with DMEM to 20 μg/mL NP concentration and directly used for cell transfection or determination of complexation efficacy. Complexation efficacy was determined indirectly from Cy5 tagged-siRNA after separating NPs and non-complexed siRNA by centrifugation (4° C., 14000 g, 15 min), quantifying Cy5 fluorescence in three replicates of the supernatant. Concentration of siRNA was determined relative to a standard curve.

High-Throughput siRNA Transfections

HeLa-GFP (CellBiolabs Inc.) cells were cultured DMEM (without phenol red) containing FBS (10%, v/v), PenStrep (0.5%, v/v, 50 μg/mL) and blasticidin (10 μg/mL). HeLa-GFP cells were seeded 24 h prior to experiment in 96 well plates (Costar) with a density of 4.000 cells per well. Cells were transfected with NP@siRNA complexes (20 μg/mL) or lipofectamine RNAiMAX (1.5 μL/ml) in DMEM as described above. Transfections were performed with three technical replicates and each plate in duplicate. After 10 min of transfection, the medium was replaced by DMEM containing 5% FBS (v/v, to slow down cell proliferation), PenStrep (0.5%, v/v, 50 μg/ml) and blasticidin (10 μg/ml). In one plate, NPs were activated for 10 min (using 365 nm light from top on a transilluminator UVP BioSpectrum 500 at 15 cm distance), while with the second plate (with the same NP formulations as the first plate), no activation of NPs was performed. This experiment allowed to compare the bioactivity of released siRNA with and without application of the stimulus. At 48 h, cells were stained and placed in an automated incubator (Cytomat 2, Thermo) for further incubation and analyses by high-content imaging with an automated fluorescence microscope (In Cell 2200, GE Healthcare).

High-Content Imaging Analyses

Cell nuclei were stained at 48 h with Hoechst H33342 (Sigma-Aldrich, 0.25 μg/mL) and propidium iodide (PI, Sigma-Aldrich, 0.25 μg/mL). Dead cells stained for both Hoechst H33342 and PI, while live cells stained only for Hoechst H33342. At 48 h and 72 h, four random fields per well were imaged on a high-content microscope (In Cell 2200, GE Healthcare) with a 20× objective. Automated image analyses were performed using the In Cell Developer software from GE Healthcare. GFP knockdown was accessed from the mean GFP fluorescence intensity in the cytoplasm of live cells. Hoechst 33342 was used to define a nuclear mask, excluding dead cells (with 10% overlap of PI and H33342 stain), which was then dilated to cover as much of the cytoplasmic region as possible. Removal of the original nuclear region from the dilated mask creates a ring mask that covers the cytoplasmic region outside the nuclear envelope. GFP knockdown was expressed as percentage of fluorescence on non-treated HeLa-GFP cells (after subtracting fluorescence background of HeLa cells). Cell viability was calculated from the total number of cells (quantified by cell nuclei) after subtraction of dead cells (cells presenting >10% overlap of PI and Hoechst stain). Internalization of NPs was quantified by the fluorescence signal of NPs in cells.

Cellular Internalization of NPs

NP@siRNA complexes were exposed for 1 h to human dermal keratinocytes (HaCaT cells; CLS Cell Lines Service GmbH, Eppelheim, Germany), human normal dermal fibroblast (NHDF) or human umbilical vein endothelial cells (HUVEC, Lonza) and characterized by flow cytometry or confocal microscopy. For flow cytometry, HaCaT and NHDF cells were cultured in DMEM medium while HUVECs were cultured in EGM-2 medium (Lonza). All media was supplemented with FBS (10%, v/v) and PenStrep (0.5%, v/v, 50 μg/mL). Cells were seeded in 24 well plates (HaCaT and HUVECs at 25.000 cells/well while NHDF cells at 12.000 cells/well) and allowed to adhere for 24 h. Cells were transfected for 1 h with NP@siRNA-Cy5 or lipofectamine@siRNA-Cy5 complexes in DMEM or EGM-2 media. Complexes were removed and cells were washed with PBS. For confocal microscopy, HaCaT and NHDF cells were cultured in DMEM medium while HUVECs were cultured in EGM-2 medium.

All media was supplemented with FBS (10%, v/v) and PenStrep (0.5%, v/v, 50 μg/ml). Cells (HaCaT and HUVECs: 20000 cells/well; NHDF cells: 10.000 cells/well) were seeded in black glass bottom 96 well plates (IBIDI, Germany) coated with 0.1% gelatin (Sigma) and allowed to adhere for 24 h. Prior to transfection cells were stained with CellTrace™ CFSE 488 (5 μM; Molecular Probes, Life Technologies) according to manufacturer's instructions. Cells were transfected for 1 h with NP@siRNA-Cy5 complexes in DMEM (for HaCaT or NHDF cells) or EBM-2 (for HUVECs). Cells were stained with Lysotracker Red (100 nM; Molecular Probes, Life Technologies) for 30 min during cell transfection. Complexes were removed and cells were washed twice with PBS and fixated with 4% (v/v) paraformaldehyde (Alfa Aesar) in PBS for 10 min at room temperature (20-30° C.). Nuclei were stained with H33342 (2 μg/mL) for 10 min. Cells were then washed 3 times with PBS and analysed by confocal microscopy (Zeiss LSM710) using a 40× immersion oil objective. Each condition is represented by two technical replicates and four or more representative images per field were acquired. Colocalization of NP@siRNA-Cy5 with Lysotracker red was performed using JaCoP on Image J.

Uptake of P1C7@siRNA-Cy5 in the Presence of Chemical Inhibitors

HeLa cells were plated in a 24 well plate at a density of 5×104 cells/well and left to adhere overnight. Cells were pre-incubated with endocytosis inhibitors for 30 min followed by 1 h incubation with P1C7@siRNA-Cy5 (20 μg/mL). The following inhibitors were tested: filipin III (80 μM), nocodazole (3 μM), polyinosinic acid (100 μg/mL), dansylcadaverine (25 μM) cytochalasin D (1 μM), dynasore (30 μM) and EIPA (80 μM). As controls, it was used cells without NPs and cells incubated with NPs without inhibitor at 37° C. and at 4° C. At the end of each point, cells were centrifuged, washed three times with PBS and then resuspended for flow cytometry analysis.

Intracellular Trafficking and siRNA Release

Endosomal escape can be determined by galectin 8 recruitment [23]. A7r5-Gal8YFP [24] reporter cells (kindly donated by Craig Duvall's lab) were used to study the colocalization of the Cy5 signal from the NP@siRNA-Cy5 complexes with YFP-Gal8 spots from releasing endosomes [23]. A7r5-Gal8YFP cells were cultured in DMEM supplemented with FBS (10%, v/v), PenStrep (0.5%, v/v, 50 μg/mL) and blasticidin (10 μg/mL). For the experiment 4.000 cells were seeded in each well of a black 96 well plate with glass bottom (IBIDI), suitable for confocal microscopy and allowed to adhere overnight. Cells were transfected for 10 min with NPs (20 μg/mL) or L2000 complexes with siRNA-Cy5 in DMEM. Cells were then washed, and cell culture medium was added to the cells. UV light (365 nm, 1 mW/cm²) activation was performed for 10 min followed by cell culture. Cells were fixated (4% PFA for 10 min) at different times (t=−10 immediately after transfection, t=0 after light activation and t=+15, 30. 45, 60 min post light activation), washed with PBS, cell nuclei stained with H33342 (1 μg/mL, Sigma) and analysed by a confocal microscope (Zeiss LSM710, 40× immersion oil objective). Each condition is represented by two technical replicates and 4 or more images per field were acquired representing the total cell population. The area of YFP-Gal8 and Cy5-NPs spots were analysed with the Particle Plugin from ImageJ. Colocalization of Cy5-labelled NPs with YFP-Gal8 spots was performed using JaCoP on Image J.

Complexation of miR150 to the NPs

The complexation of miR150 (GE Dharmacon) to P1C7 NPs followed the same procedure previously described for siRNA. Briefly, miR150 and P1C7 NPs were mixed in molecular grade nuclease free, sterile water (Fisher Bioreagents) in a ratio of 1:50 (w/w, miRNA to NPs), and the suspension agitated on an orbital shaker for 2 h at room temperature (20-30° C.). After complexation, the NP suspension was diluted in cell culture medium before use.

Bioactivity of P1C7@miRNA150 Formulation

Human keratinocytes were cultured in DMEM supplemented with 10% FBS and 0.5% PenStrep, harvested and then seeded in a 96 well plate at a density of 25.000 cells/well to grow to a monolayer in approximately 48 h. Cells were then inhibited by mitomycin (5 μg/mL, in cell culture medium, Tocris Bioscience) for 2 h, transfected for 4 h with P1C7 NPs (40 μg/mL in DMEM) or P1C7 NPs@miRNA150 complexes (40 μg/mL in DMEM), washed with PBS to remove non-internalized NPs, exposed or not to UV light (10 min, 365 nm, 1 mW/cm2). The cell monolayer was then scratched with a pipette tip and then cultured in cell culture medium for 48 h. As controls, cells were transfected for 4 and 24 h with the commercial agent lipofectamine RNAiMAX or RNAiMAX-miR150 complexes. In both cases (NPs and lipofectamine), wound closure was monitored by an automated fluorescence microscope (In Cell 2000, GE Healthcare, 4× objective) every 12 h. Wound closure was analyzed from the image field in the center of the well, measuring the wound area with Image J. The percentage of wound closure was calculated by well considering the initial wound area and then normalized to the control of the respective group.

Quantitative Analyses of miR150 Transfection

To demonstrate that HaCaT cells were successfully transfected with P1C7 NPs@miRNA150 complexes, cells were transfected with P1C7 NPs or P1C7 @miRNA150 NPs for 4 h, washed with PBS to remove non-internalized NPs, light activated, and finally cultured for 48 h. Next, cells were harvested, lysed and RNA isolated by a miRCURY™ RNA isolation kit (Exiqon) following manufacturer's instructions. The cDNA was then synthesized using the Mir-X™ miRNA First Strand Synthesis (Exiqon). Expression of miRNA was quantified by quantitative RT-PCR (7500 Fast Real-Time PCR System, Applied Biosystems, Carlsbad, Calif., USA) using Mir-X™ SYBR qRT-PCR kit (Clontech, California, USA) and NZYSpeedy qPCR Green Master Mix (NZYTech, Portugal). For normalization of microRNA expression levels, RNU6 was used as (housekeeping) control (FIG. 18, Table 3). Results were analyzed using the ΔΔC_{T} method to indicate relative miR150 expression from light activated miR-carrying to non-carrying P1C7 NPs.

Quantitative Analysis of Target Gene Knockdown

HaCaT cells transfected with P1C7 NPs or P1C7@miRNA150 NPs (see section before) were analyzed for the expression of cMYB, a target gene of miR150. Therefore, cDNA was synthesized from 1 μg total RNA using TaqMan™ reverse transcription reagents (Applied Biosystems, CA, USA). Quantitative RT-PCR was performed using NZYSpeedy qPCR Green Master Mix (NZYTech, Portugal) on a RT-PCR (7500 Fast Real-Time PCR System, Applied Biosystems, Carlsbad, Calif., USA). Quantification of the target gene was analyzed relative to GAPDH as housekeeping gene: relative expression=2^{[-(C}^{_{T Sample}}^{-C}^{_{T GAPDH}}^{)]}. Minimal cycle threshold values (CT) were calculated from at least 3 independent reactions. ΔΔC_{T} was calculated to determine relative cMYB expression from light activated miR-carrying to non-carrying P1C7 NPs.

In Vivo Wound Healing Experiments

Animal protocol was approved by the Ethics Committee of the Faculty of Medicine of the University of Coimbra (ORBEA_159_2017/05052017). Male C57BL/6 mice (8 weeks) were purchased from Charles River (Wilmington, Mass., USA). Mice were separated on individual cages 24 h prior the induction of the skin wounds. They were anesthetized with xylazine/ketamine (xylazine hydrochloride—Rompun, 10 mg/kg of body weight; ketamine hydrochloride—Imalgene 1000, 80 mg/kg of body weight), shaved with an electric clipper on the back and remaining hair removed with depilatory cream (Dove), the skin was disinfected with betadine and two 6 mm-diameter dorsal full-thickness excisional wounds were created with a sterile biopsy punch in each animal. The treatments (P1C7@miRNA150, P1C7@scramble, PBS; n=8) were administered as intradermal injection at 4 locations around the wound. Light (5 min; 5 sec on/off; 405 nm blue laser; Thorlabs, Dachau, Germany) activation of the NPs was performed 30 min post-injection, to allow the NPs to be internalized by the skin cells. During the first 2 days, the mice received every 8 h buprenorphine (0.05 mg/Kg of body weight, Bupaq) to relieve the animals from any pain or distress caused by the procedure. The animals were observed daily, and wound area was measured. At day 3 and 10 mice were sacrificed by cervical dislocation after an overdose of anesthesia. Skin biopsies were taken for histological and gene expression analyses.

Histological Analyses of Skin Wounds

Skin wounds were excised with a margin of epidermis outside the wound (approx. 2 mm) and processed for routine histology. Therefore, freshly excised wounds were placed onto a small piece of cardboard, with the subcutaneous tissue facing down, and immersed in 10% neutral buffered formalin for 24 h. After fixation, trimming was performed longitudinally, in the direction of the hair flow and centered on the wound. Samples were embedded in paraffin, sectioned at 4 μm, and stained with hematoxylin and eosin. Histological analysis was performed by a pathologist blinded to experimental groups and measurements were performed using NDP.view2 software coupled to Nanozoomer SQ slide scanner (Hamamatsu).

Quantitative Analysis of miRNA150 Target Gene in Skin Wounds

To quantify downregulation of miR150 gene targets in the skin wounds, samples at day 3 post-surgery were analyzed. RNA was isolated using TRIzol (500 μL, Invitrogen) reagent from 15-50 mg of tissue. Samples on ice were homogenized by a TissueLyser (Qiagen) operated at 30 Hz in three cycles of 2 min. Samples were then processed according to manufacturer's instructions for RNA isolation and RNA was quantified on a NanoDrop™ (Thermo Scientific). cDNA was prepared from 1 μg total RNA using TaqMan™ reverse transcription reagents (Applied Biosystems, CA, USA). Quantitative RT-PCR (qRT-PCR) of murine cMYB was performed using NZYSpeedy qPCR Green Master Mix (NZYTech, Portugal) and detection on a RT-PCR (7500 Fast Real-Time PCR System, Applied Biosystems, Carlsbad, Calif., USA) equipment. Quantification of the target gene was analyzed relative to mouse GAPDH as housekeeping gene: relative expression=2^{[-(C}^{_{T Sample}}^{-C}^{_{T GAPDH}}^{)]}. Minimal cycle threshold values (CT) were calculated from at least 3 independent reactions. ΔΔC_{T} was calculated to determine downregulation of cMYB relative to control skin (tissue day 0).

Light Activation (i.e. Exposure to Light to Promote NP Disassembly) of P1C7 NPs Though a Skin Barrier

To demonstrate that NPs can be dissociated by a 405 nm laser through the skin barrier, back skin from C57BL/6 mice was used as barrier between the light source and the NPs. Briefly, hair was removed from mouse skin with depilation cream and washed several times with PBS and mounted on a cardboard plate with a 1 cm² hole with the geometry of DLS cuvettes. A 405 nm laser (Thorlabs, Germany) was used for the experiment. Laser power intensity by cm² was measured with a digital optical power and energy meter (Thorlabs, Germany) with and without the skin barrier (skin thickness 0.26-0.29 mm). P1C7 NPs (50 μg/mL) were activated for 10 min with the laser with or without the skin barrier and then measured by DLS. Laser power transmission and NP count decrease were analyzed for 3 individual samples.

The terms “light triggerable” or “light sensitive” in the context of the present application mean compounds or molecules which physical or chemical structures or properties change when exposed to radiation, i. e. light.

The term “library” in the context of the present application means a collection of compounds or formulations with similarities in their chemical composition and having the same purpose of application.

The term “light activation” or “activated” in the context of the present application means exposing the polymeric nanoparticles to light and that the disclosed nanoparticles are disassembled when exposed to incident light by the photocleavage of a side hydrophobic group or by the photocleavage of the photocleavable linker that is integrated in the polymeric nanoparticles.

### REFERENCES

1. Juliano, R. L., The delivery of therapeutic oligonucleotides. Nucleic Acids Res, 2016. 44(14): p. 6518-48.
2. Dowdy, S. F., Overcoming cellular barriers for RNA therapeutics. Nat Biotechnol, 2017. 35(3): p. 222-229.
3. Gilleron, J., et al., Image-based analysis of lipid nanoparticle-mediated siRNA delivery, intracellular trafficking and endosomal escape. Nat Biotechnol, 2013. 31(7): p. 638-46.
4. Kanasty, R., et al., Delivery materials for siRNA therapeutics. Nat Mater, 2013. 12(11): p. 967-77.
5. Wittrup, A., et al., Visualizing lipid-formulated siRNA release from endosomes and target gene knockdown. Nat Biotechnol, 2015. 33(8): p. 870-6.
6. Huschka, R., et al., Gene Silencing by Gold Nanoshell-Mediated Delivery and Laser-Triggered Release of Antisense Oligonucleotide and siRNA. ACS Nano, 2012. 6(9): p. 7681-7691.
7. Chang, Y.-T., et al., Near-Infrared Light-Responsive Intracellular Drug and siRNA Release Using Au Nanoensembles with Oligonucleotide-Capped Silica Shell. Advanced Materials, 2012. 24(25): p. 3309-3314.
8. Yang, Y., et al., NIR light controlled photorelease of siRNA and its targeted intracellular delivery based on upconversion nanoparticles. Nanoscale, 2013. 5(1): p. 231-238.
9. Braun, G. B., et al., Laser-Activated Gene Silencing via Gold Nanoshell-siRNA Conjugates. ACS Nano, 2009. 3(7): p. 2007-2015.
10. Wang, H., et al., A Near-Infrared Laser-Activated “Nanobomb” for Breaking the Barriers to MicroRNA Delivery. Advanced Materials, 2016. 28(2): p. 347-355.
11. Sahay, G., et al., Efficiency of siRNA delivery by lipid nanoparticles is limited by endocytic recycling. Nat Biotechnol, 2013. 31(7): p. 653-8.
12. Whitehead, K. A., et al., Degradable lipid nanoparticles with predictable in vivo siRNA delivery activity. Nat Commun, 2014. 5: p. 4277.
13. Kozielski, K. L., et al., Bioreducible cationic polymer-based nanoparticles for efficient and environmentally triggered cytoplasmic siRNA delivery to primary human brain cancer cells. ACS Nano, 2014. 8(4): p. 3232-41.
14. Dahlman, J. E., et al., In vivo endothelial siRNA delivery using polymeric nanoparticles with low molecular weight. Nat Nanotechnol, 2014. 9(8): p. 648-655.
15. Gehin, C., et al., Dynamic amphiphile libraries to screen for the “fragrant” delivery of siRNA into HeLa cells and human primary fibroblasts. J Am Chem Soc, 2013. 135(25): p. 9295-8.
16. Zhou, Y., et al., Photoresponsive Drug/Gene Delivery Systems. Biomacromolecules, 2018.
17. Klan, P., et al., Photoremovable protecting groups in chemistry and biology: reaction mechanisms and efficacy. Chem Rev, 2013. 113(1): p. 119-91.
18. Lucas, T., et al., Light-inducible antimiR-92a as a therapeutic strategy to promote skin repair in healing-impaired diabetic mice. Nat Commun, 2017. 8: p. 15162.
19. Lino, M. M., et al., Modulation of Angiogenic Activity by Light-Activatable miRNA-Loaded Nanocarriers. ACS Nano, 2018.
20. Akinc, A., et al., Synthesis of poly(beta-amino ester)s optimized for highly effective gene delivery. Bioconjug Chem, 2003. 14(5): p. 979-88.
21. Kilchrist, K. V., et al., Mechanism of Enhanced Cellular Uptake and Cytosolic Retention of MK2 Inhibitory Peptide Nano-polyplexes. Cell Mol Bioeng, 2016. 9(3): p. 368-381.
22. Xiao, C., et al., MiR-150 controls B cell differentiation by targeting the transcription factor c-Myb. Cell, 2007. 131(1): p. 146-59.
23. Wittrup, A., et al., Visualizing lipid-formulated siRNA release from endosomes and target gene knockdown. Nat Biotechnol, 2015. 33(8): p. 870-6.
24. Kilchrist, K. V., et al., Mechanism of Enhanced Cellular Uptake and Cytosolic Retention of MK2 Inhibitory Peptide Nano-polyplexes. Cell Mol Bioeng, 2016. 9(3): p. 368-381.

1. A light-triggerable nanoparticle library of formulations for controlled release of RNAs wherein the formulations comprise:
polymeric nanoparticles comprising photocleavable linker monomers;
amine monomers; and
bisacrylamide monomers;
wherein the nanoparticles are complexed with RNA; and
wherein the nanoparticles are adapted to be disassembled when exposed to light.

2. The light-triggerable nanoparticle library of formulations according to claim 1, wherein the photocleavable linker is 2-nitro-1,3-phenylene)bis(methylene) diacrylate (P1).

3. The light-triggerable nanoparticle library of formulations according to claim 1, wherein the bisacrylamide monomers are selected from methylenebisacrylamide (A), hexamethylenebisacrylamide (B), cystaminebisacrylamide (C), dihydroxyethylenebisacrylamide (D), or bisacryloylpiperazin (E).

4. The light-triggerable nanoparticle library of formulations according to claim 1, wherein the amine monomers are selected from the group consisting of ethylenediamine (1), 1,4-diaminobitan (2), 1,6-diaminohexan (3), diethylenetriamine (4), triethylenetetramine (5), pentaethylenehexamine (6), 3,3′-diamino-N-methyldipropylamine (7), 1,2-diaminocyclohexane (8), 1,8-diamino-3,6-dioxoctane (9), 1,13-diamono-4,7,10-trioxatridecane (10), 1,4-bis(aminopropyl)piperazine (11), 1,4-phenylenedimethanamine (12), 1,5-diaminonaphthalene (13), 4,4′-methylenedianiline (14), 1,3-phenylenediamine (15), 1,3-diaminopropane (16), 2,2-dimethyl-1,3-propanediamine (17), 1,3-diamiopentane (18), 2,2′-diamino-N-methyldiethylamine (19), agmatine sulfate (20), 1,4-Bis(aminomethyl)cyclohexane (21), 4,4′-methylenebis(cyclohexylamine) (22), 4,4′-diaminobenzanilide (23), DL-Lysine (24), 3-amino-1-propanol (25), 4-amino-1-butanol (26), 5-amino-1-pentanol (27), 6-amino-1-hexanol (28), 1-(3-aminopropyl)pyrrolidine (29), 1-(3-aminopropyl)imidazole (30), 1-(3-aminopropyl)-4-methylpiperazine (31), and histamine (32).

5. The light-triggerable nanoparticle library of formulations according to claim 1, wherein a maximum percentage molar ratio is between 21% and 23% of P1, between 21% and 23% of bisacrylamide and between 54% and 58% of amines.

6. The light-triggerable nanoparticle library of formulations according to claim 1, wherein a molar ratio of P1 per repeating unit of the polymer is 25 (P1):25(bisacrylamide):50(amine).

7. The light-triggerable nanoparticle library of formulations according to claim 1, wherein 90% of the nanoparticles have a size range between 100 and 500 nm.

8. The light-triggerable nanoparticle library of formulations according to claim 1, wherein 20% of the nanoparticles have a zeta potential above 20 mV.

9. The light-triggerable nanoparticle library of formulations according to claim 1, wherein 80% of the formulations show 50% count decrease after 10 min of light exposure.

10. The light-triggerable nanoparticle library of formulations according to claim 1, wherein a ratio of siRNA:NP or miRNA:NP is 1:50.

11. The light-triggerable nanoparticle library of formulations according to claim 1, wherein a ratio of mRNA:NP (w/w) varies between 1:5 and 1:100.

12. The light-triggerable nanoparticle library of formulations according to claim 1, comprising formulations of P1A1, P1A7, P1C5, P1C7.

13. The light-triggerable nanoparticle library of formulations according to claim 1, wherein they have a cell transfection time of 10 minutes or less.

14. The light-triggerable nanoparticle library of formulations according to claim 1, wherein the nanoparticles have a complexation efficiency with RNA between 75 and 125%.

15. A process to produce a light-triggerable nanoparticle library of formulations for controlled release of RNAs according to claim 1 comprising the following steps:
reacting the monomers in a molar ratio of 25(P1):25(bisacrylamide):50(amine);
end capping the polymers with 20% molar excess of the respective amine 1-32, wherein the amine monomers are selected from the group consisting of ethylenediamine (1), 1,4-diaminobitan (2), 1,6-diaminohexan (3), diethylenetriamine (4), triethylenetetramine (5), pentaethylenehexamine (6), 3,3′-diamino-N-methyldipropylamine (7), 1,2-diaminocyclohexane (8), 1,8-diamino-3,6-dioxoctane (9), 1,13-diamono-4,7,10-trioxatridecane (10), 1,4-bis(aminopropyl)piperazine (11), 1,4-phenylenedimethanamine (12), 1,5-diaminonaphthalene (13), 4,4′-methylenedianiline (14), 1,3-phenylenediamine (15), 1,3-diaminopropane (16), 2,2-dimethyl-1,3-propanediamine (17), 1,3-diamiopentane (18), 2,2′-diamino-N-methyldiethylamine (19), agmatine sulfate (20), 1,4-Bis(aminomethyl)cyclohexane (21), 4,4′-methylenebis(cyclohexylamine) (22), 4,4′-diaminobenzanilide (23), DL-Lysine (24), 3-amino-1-propanol (25), 4-amino-1-butanol (26), 5-amino-1-pentanol (27), 6-amino-1-hexanol (28), 1-(3-aminopropyl)pyrrolidine (29), 1-(3-aminopropyl)imidazole (30), 1-(3-aminopropyl)-4-methylpiperazine (31), and histamine (32);
preparing nanoparticles (NPs) by precipitation of the polymers in sterile nuclease free molecular grade water and zinc sulfate;
complexing RNAs with the NPs.

16. A method for controlled release of RNAs in skin, eyes and intestines of a subject in need thereof comprising applying the light-triggerable nanoparticle library of formulations according to claim 1.
